SEQUENCE LISTING (2 sequences)

SEQ ID NO: 1 - protein, 478 aa, Homo sapiens
NPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 2 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

(19) United States
(12) Patent Application Publication
(10) Pub. No.: US 2015/0224205 A1
(43) Pub. Date: Aug. 13, 2015

(54) POLYMERIC CONJUGATES OF C-1 INHIBITORS

(71) Applicant: Belrose Pharma, Inc., Princeton, NJ (US)

(72) Inventors: Hong Zhao, Edison, NJ (US); Karen Yang, Edison, NJ (US)

(21) Appl. No.: 14/358,983

(22) PCT Filed: Mar. 15, 2013
(86) PCT No.: PCT/US2013/031930

§ 371 Date: May 16, 2014

Related U.S. Application Data
(60) Provisional application No. 61/612,213, filed on Mar. 16, 2012.
(60) Provisional application No. 61/749,840, filed on Jan. 7, 2013.
(60) Provisional application No. 61/749,842, filed on Jan. 7, 2013.
(60) Provisional application No. 61/749,848, filed on Jan. 7, 2013.

(51) Int. Cl. A61K 47/48; C07K 14/81

(52) U.S. Cl. CPC A61K 47/48215; C07K 14/8121; A61K 38/00

(57) ABSTRACT

Polymer conjugates containing a C1-inhibitor having at least one substantially non-antigenic polymer covalently attached to the C1-inhibitor via amino group of the C1 inhibitor are provided. In the polymer conjugates, the substantially non-antigenic polymer is attached to the N-terminal of C1-inhibitor. Alternatively, the substantially non-antigenic polymer is attached to the N-terminal of C1-inhibitor and at least one more of the substantially non-antigenic polymer is attached to lysine and/or histidine of the C1-inhibitor. Furthermore, the polymer conjugates is attached to C1-inhibitor either via permanent or releasable spacers. In addition, methods of making the conjugates as well as methods of treatment using the conjugate of the present invention are also provided.

### CROSS-REFERENCE TO RELATED APPLICATIONS

This application claims the benefit of priority from U.S. Provisional Patent Application Ser. Nos. 61/612,213 filed Mar. 16, 2012, and 61/749,840, 61/749,842 and 61/749,848 filed Jan. 7, 2013, the contents of each of which are incorporated herein by reference.

### FIELD OF INVENTION

The present invention relates to polymeric conjugates containing a C1-inhibitor having at least one substantially non-antigenic polymer covalently attached to the C1-inhibitor via an amino group of the C1 inhibitor and uses thereof

### BACKGROUND OF THE INVENTION

C1-inhibitor is a normal constituent of human plasma and belongs to the group of serine protease inhibitors (serpins), One type of C1-inhibitor, C1 esterase inhibitor, is a soluble, single-chain glycoprotein containing 478 amino acid residues. The plasma concentration of C1-esterase inhibitor in a healthy human body is approximately 270 mg/L.

C1-inhibitor is a down-regulator of inflammatory processes in blood. Unlike most family members, C1-inhibitor has a 2-domain structure: the C-terminal serpin domain, which is similar to other serpins, and the N-terminal domain. Structural analysis showed the N-terminal is highly glycosylated leaving the C-terminal more susceptible to reactive binding sites.

Deficiency of this protein is associated with hereditary angioedema or angioneurotic edema, or swelling due to leakage of fluid from blood vessels into connective tissue. Symptoms include swelling of the face, mouth and/or airway that occurs spontaneously or by minimal triggers (such as mild trauma). Such swelling can also occur in any part of the body. In some cases, the levels of C1-inhibitor are low, while in others the protein circulates in normal amounts but it is dysfunctional. In addition to the episodes of facial swelling and/or abdominal pain, it also can cause more serious or life threatening indications, such as autoimmune diseases or lupus erythematosus.

In people with hereditary angioedema, Cinryze® is used to prevent attacks of angioedema, when the C1-esterase inhibitor does not function properly or occurs in low levels, while Berinert® is used to treat attacks of angioedema. Cinryze® is administered at a dose of 1,000 units intravenously at 1 mL/min for 10 min, every 3 or 4 days for routine prophylaxis against angioedema attacks, and Berinert® is administered at a dose of 20 units per kg body weight intravenously at 4 mL/min. Accordingly, non-compliance is a major Obstacle to the effective delivery of the C1-esterase inhibitor.

In spite of previous efforts, there is still an unmet need for an improved form of a C1-inhibitor. For example, it would be beneficial to provide long acting C1-inhibitors so that the to frequency of dosing could be reduced. The present invention addresses this need.

### SUMMARY OF THE INVENTION

Accordingly, in order to provide the desired improvements, the present invention provides a polymer conjugate containing a C1-inhibitor having at least one substantially non-antigenic polymer covalently attached to the C1-inhibitor via an amino group of the C1 inhibitor. Another aspect of the invention, polymer conjugates are provided in which one of the substantially non-antigenic polymer is attached to the N-terminal of C1-inhibitor. In another aspect of the invention, polymer conjugates are provided in which one of the substantially non-antigenic polymer is attached to the N-terminal of C1-inhibitor and at least one more of the substantially non-antigenic polymer is attached to lysine and/or histidine of the C1-inhibitor via a permanent or a releasable linkers.

Methods of making the conjugates as well as methods of treatment using the conjugate of the present invention are also provided. Advantages will be apparent from the following description.

For purposes of the present invention, the term “residue” shall be understood to mean that portion of a conjugate, to which it refers, e.g., amino acid, etc. that remains after it has undergone a substitution reaction with another conjugate.

For purposes of the present invention, the term “polymeric containing residue” or “PEG residue” shall each be understood to mean that portion of the polymer or PEG which remains after it has undergone a reaction with C1-inhibitor.

For purposes of the present invention, the term “alkyl” shall be understood to include straight, branched, substituted, e.g. halo-, alkoxy-, nitro-, C₁₋₁₂, but preferably C₁₋₄ alkyls, C₃₋₈ cycloalkyls or substituted cycloalkyls, etc.

For purposes of the present invention, the term “substituted” shall be understood to include adding or replacing one or more atoms contained within a functional group or conjugate with one or more different atoms.

For purposes of the present invention, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

The terms “effective amounts” and “sufficient amounts” for purposes of the present invention shall mean an amount which achieves a desired effect or therapeutic effect as such effect is understood by those of ordinary skill in the art.

### DETAILED DESCRIPTION OF THE INVENTION

The present invention provides a polymer conjugate of a C1-inhibitor having at least one substantially non-antigenic polymer covalently attached thereto.

In one embodiment, polymer conjugates are provided in which the substantially non-antigenic polymer is a polyalkylene oxide. In another embodiment, polymer conjugates are provided in which the polyalkylene oxide is polyethylene glycol.

In yet another embodiment, polymer conjugates are provided where the C1-inhibitor is a human C1 esterase inhibitor (C1-INH) or a polypeptide represented by SEQ ID NO: 1 or SEQ ID NO:2.

In a further embodiment, polymer conjugates are provided in which one of the substantially non-antigenic polymer is attached to the N-terminal of the C1-inhibitor.

In one aspect of the invention, polymer conjugates are provided in which one of the substantially non-antigenic polymer is attached to an epsilon amino group of a lysine. In another aspect of the invention, polymer conjugates are provided in which one of the substantially non-antigenic polymer is attached to histidine of the C1-inhibitor.

In yet another aspect of the invention, polymer conjugates are provided in which the polymer conjugate further comprises at least one substantially non-antigenic polymer attached to the N-terminal and another polymer attached to an epsilon amino group of a lysine.

In a further aspect of the invention, polymer conjugates are provided in which the polymer conjugate further comprises at least one substantially non-antigenic polymer attached N-terminal and another polymer attached to an epsilon amino group of a lysine and/or histidine of the C1-inhibitor.

The polymer conjugates of the invention retain about 20-80% of the biological activity of the native C1-inhibitor. Preferably, the polymer conjugates of the invention retain about 40-80% of the biological activity of the native C1-inhibitor.

In a further embodiment, polymer conjugates are provided in which the polymer conjugate has the formula (I):

[PEG—(L)ₘ—(CH₂)ₙ]ₚ—(X)_{p′}—C1-inhibitor (I)
wherein
PEG is linear, branched or multi-arm PEG having terminal group —(CH₂CH₂O)—;
L is a linker;
(m) 0 or 1;
(n) is zero or a positive integer, preferably selected from 1, 2, 3, 4, 5, 6, 7, 8, 9, or 10;
(p) is a positive integer, preferably selected from 1, 2, 3, 4, 5, 6 or 7, and more preferably is less than or equal to the number of available amine residues or lysine groups on the C1-Inhibitor which are available; and
X is an amine group of an amino acid found on C1-inhibitor attached to the polymer; (p′) is a positive integer same as (p), provided that (m) and (n) are not zero simultaneously.

In one aspect of the invention, in the polymer conjugate of Formula (I) described above, (n) is a positive integer selected from among 1, 2, 3, 4, 5, 6 or 7 and (p) is a positive integer selected from among of 1, 2 or 3,

In another embodiment, in the polymer conjugate of Formula (J) described above, L is selected from the group consisting of:

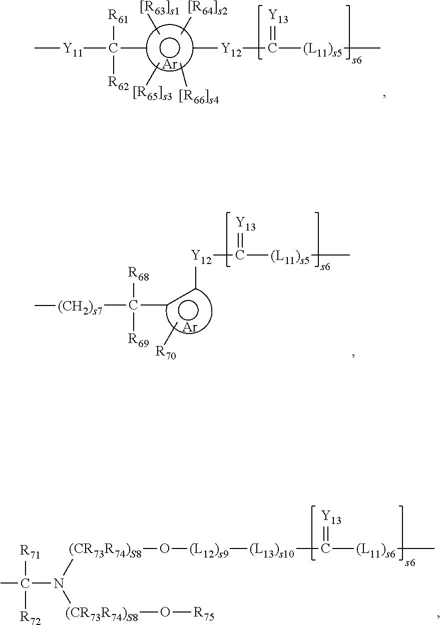

wherein
Y₁₁ is O, or S;
Y₁₂ is O, S, or NH, provided that L₁₁ is Gly-Phe-Leu-Gly, Ala-Leu-Ala-Leu, Phe-Lys, or Val-Cit, when Y₁₂ is NH and (s6) is a positive integer;
Y₁₃ is O, S, or NR₆₇;
L₁₁₋₁₃ are independently bifunctional linking moiety selected from the group consisting of

—[C(═O)]ₛ₁₁CR₇₆R₇₇OCR₇₆R₇₇[C(═O)]ₛ₁₂—[Y₁₅]ₛ₁₃—;

—[C(═O)]ₛ₁₁CR₇₆R₇₇NR₇₈CR₇₆R₇₇[C(═O)]ₛ₁₂—[Y₁₅]ₛ₁₃—;

—[C(═O)]ₛ₁₁CR₇₆R₇₇SCR₇₆R₇₇[C(═O)]ₛ₁₂—[Y₁₅]ₛ₁₃—; and

—[C(═O)]ₛ₁₁(CR₇₆R₇₇)ₛ₁₁[C(═O)]ₛ₁₂—[Y₁₅]ₛ₁₃—; or

C(═Y₁₃)—L11— together form an amino acid;
Y₁₅ O, S or NRₓ;
(s13) is 0 or a positive integer;
R₆₁, R₆₂, R₆₇, R₇₁, R₇₂, R₇₃, R₇₄ and Rₓ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls;
R₆₃, R₆₄, R₆₅ and R₆₆ are independently selected from the group consisting of hydrogen, C₁₋₆ alkyls, C₁₋₆ alkoxy, phenoxy, C₁₋₈ heteroalkyls, heteroalkoxy, substituted C₁₋₆ alkyls, C₃₋₈ cycloalkyls, substituted cycloalkyls, aryls, substituted aryls, aralkyls, halo-, nitro-, cyano-, carboxy-, C₁₋₆ carboxyalkyls and C₁₋₆ alkyl carbonyls;
R₆₈, R₆₉ and R₇₀ are independently selected from the group consisting of C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, heteroalkyls, substituted C₁₋₆ heteroalkyls, C₁₋₆ alkoxy, phenoxy, and C₁₋₆ heteroalkoxy;
R₇₅ is H, —C(═O)-R₇₉, wherein R₇₉, in each occurrence, is the same or different alkyl,

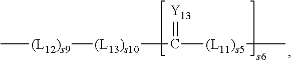

or a targeting group;
R₇₆, R₇₇ and R₇₈ are independently selected from the group consisting of from H, C₁₋₆ alkyl, C₂₋₆ alkenyl, C₂₋₆ alkynyl, C₁₋₆ heteroalkyl and aryl;
Ar is a moiety which when included in Formula (I) forms an aromatic or heteroaromatic hydrocarbon;
(s1), (s2), (s3), and (s4) are independently zero or one;
(s5) is a positive integer of from about 1 to about 6;
(s6) is zero or a positive integer;
(s7) is zero, one or two;
(s8) is 1, 2 or 3;
(s9) is zero or one;
(s10) is zero or a positive integer of from about 1 to about 6′
(s11), (s12), and (s13) are independently zero or one.

### Polymers

In one preferred embodiment, the polymer conjugate described herein can employ a variety of water soluble polymers which have the following formula:

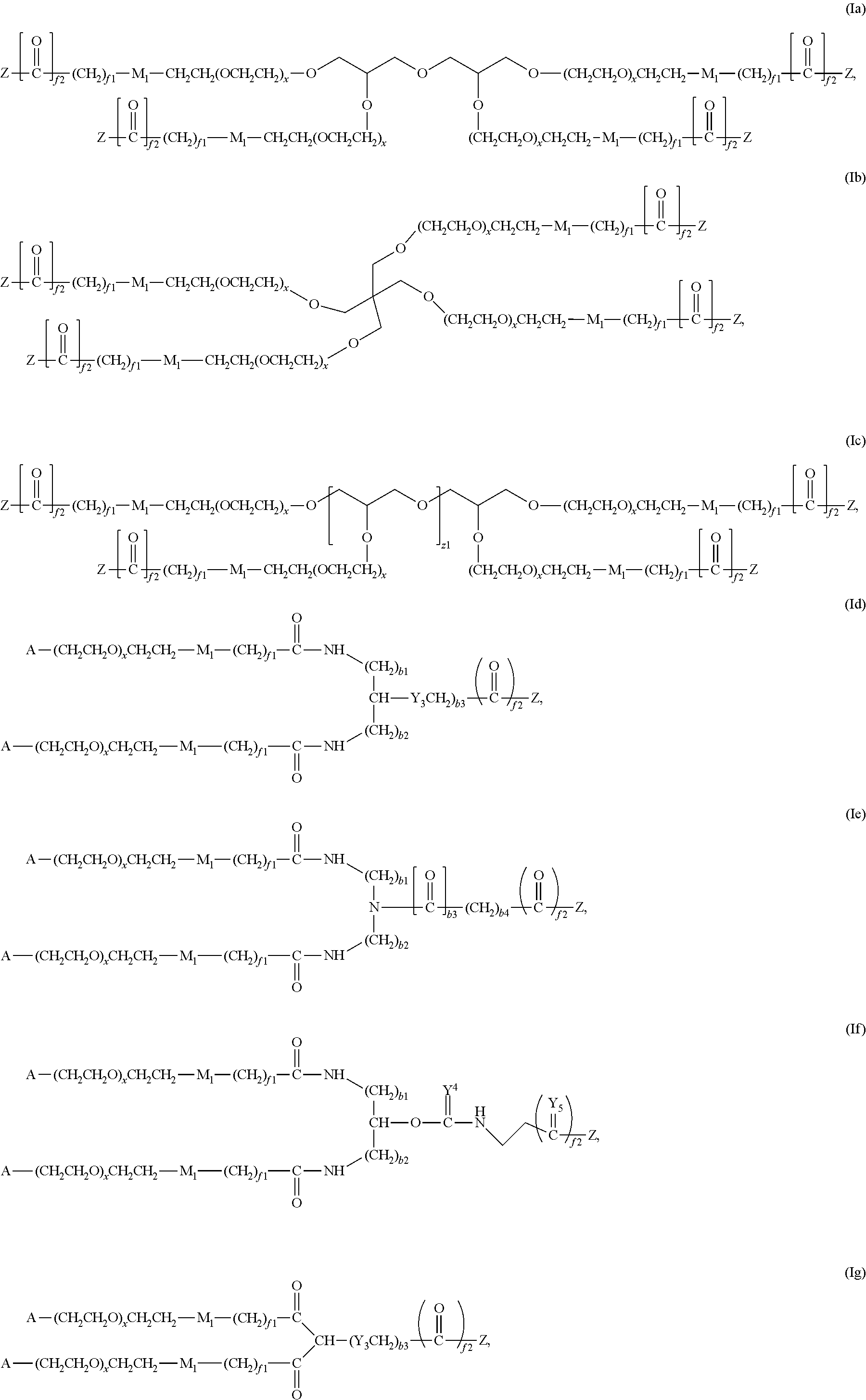

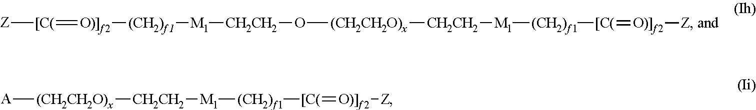

wherein
A is hydroxyl NH₂, CO₂H, or C₁₋₆ alkoxy;
M₁ is O, S, or NH;
Y₃ is O, NR₅₁, S, SO or SO₂;
Y₄ and Y₅ are independently O, S or NR₅₁;
R₅₁, is each occurrence, is independently hydrogen, C₁₋₈ alkyl, C₁₋₈ branched alkyl, C₁₋₈ substituted alkyl, aryl, or aralkyl;
Z, in each occurrence, is independently OH, a leaving group, a targeting group, C₁₋₈ alkyl, C₁₋₈ alkoxy, C1 inhibitor or C1 inhibitor containing moiety;
(b1) and (b2) are independently zero or positive integers;
(b3) is zero or 1;
(b4) is a positive integer;
(f1) is zero or a positive integer of from about 1 to about 10;
(f2) is zero or 1;
(z1) is zero or a positive integer of from 1 to about 27;
(n) is a positive integer of from about 10 to about 2,300 so that the polymeric portion of the conjugate has the total number average molecular weight of from about 2,000 to about 100,000 daltons; and
all other variables are the same as previously defined; provided that one or more Z is a C1-inhibitors or C1-inhibitor containing moiety.

In a certain embodiment, the molecular weight of the substantially non-antigenic polymer ranges from about 2,000 to about 60,000 daltons, preferably the molecular weight of the substantially non-antigenic polymer ranges from about 5,000 to about 50,000 daltons, and more preferably from about 20,000 to about 40,000 daltons.

In another embodiment, the substantially non-antigenic polymer is conjugated via a linker. In yet another embodiment, the substantially non-antigenic polymer is conjugated via amine, amide bond or carbamate bond.

According to the present invention, polymers contemplated within the conjugates described herein are preferably water soluble and substantially non-antigenic, and include, for example, polyalkylene oxides (PAO's). The conjugates described herein further include linear, branched, or multi-armed polyalkylene oxides. In one preferred aspect of the invention, the polyalkylene oxide includes polyethylene glycols and polypropylene glycols. More preferably, the polyalkylene oxide includes polyethylene glycol (PEG).

PEG is generally represented by the structure

—(CH₂CH₂O)ₓ—

where (x) is a positive integer of from about 10 to about 2300 so that the polymeric portion of the conjugates described herein has a number average molecular weight of from about 2,000 to about 100,000 daltons.

The polyalkylene oxide has a total number average molecular weight of from about 2,000 to about 100,000 daltons, preferably from about 5,000 to about 60,000 daltons. The molecular weight of the polyalkylene oxide can be more preferably from about 5,000 to about 25,000 or from about 20,000 to about 45,000 daltons. In some particularly preferred embodiments, the conjugates described herein include the polyalkylene oxide having a total number average molecular weight of from about 30,000 to about 45,000 daltons. In one particular embodiment, a polymeric portion has a total number average molecular weight of about 40,000 daltons.

Alternatively the polyethylene glycol can be farther functionalized as represented by the structure:

—[C(═O)]_{f2}—(CH₂)ₙ—M₁—CH₂CH₂(OCH₂CH₂)ₙ—O—A
wherein
M₁ is O, S, or NH;
(f1) is zero or a positive integer of from about 1 to about 10, preferably, 0, 1, 2, or 3, more preferably, zero or 1;
(f2) is zero or one;
(n) is a positive integer of from about 10 to about 2,300; and
A is hydroxyl, NH₂, CO₂H, or C₁₋₆ alkoxy.

In one embodiment, A is methoxy.

In certain embodiments, all four of the PEG arms can be converted to suitable activating groups, for facilitating attachment to other molecules (e.g., bifunctional linkers). Such conjugates prior to conversion include:

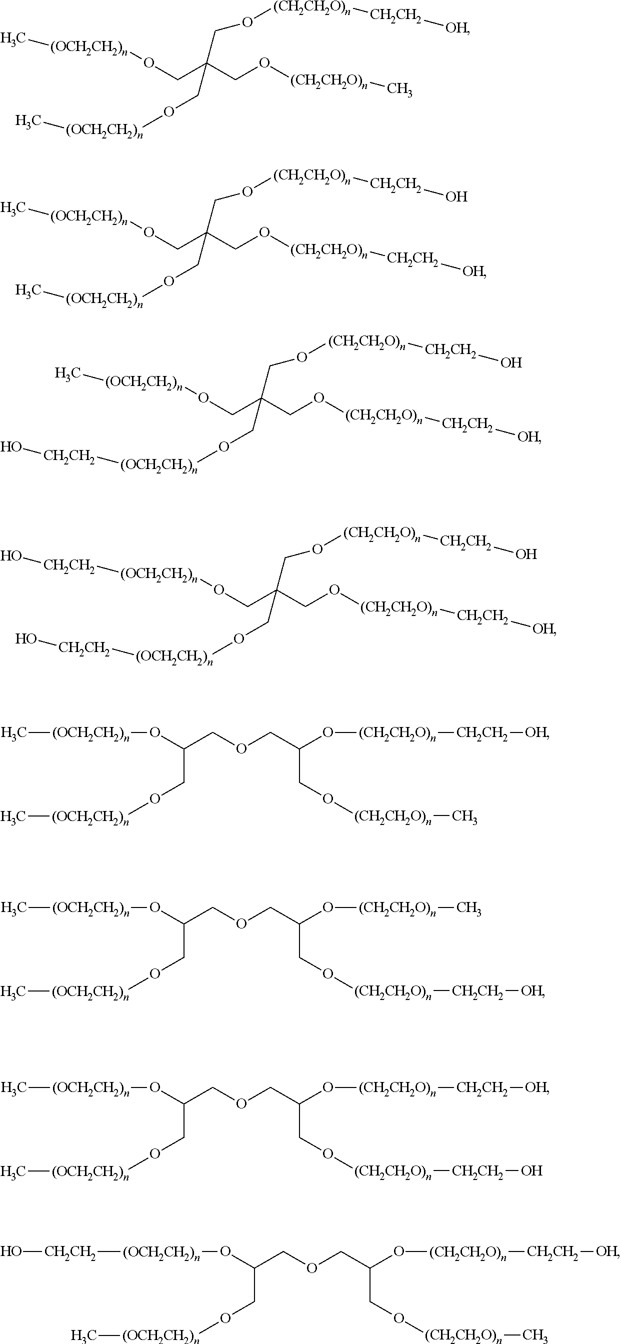

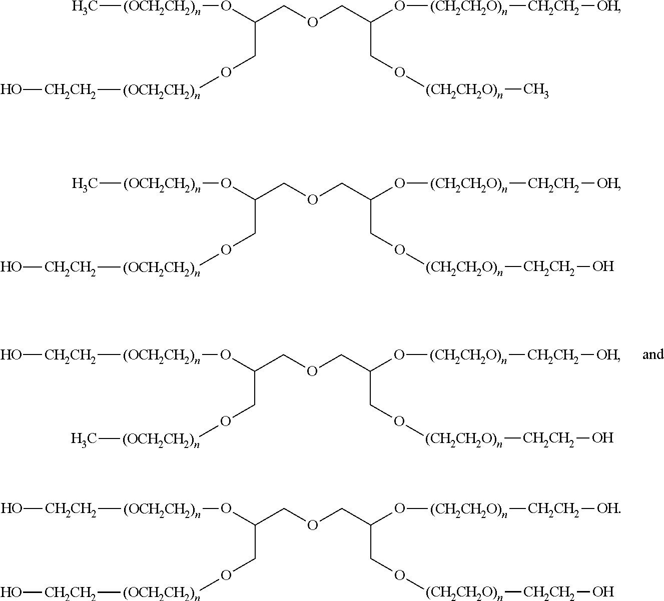

At least one, if not all PEG arms should include aldehyde or other amine PEGylating linker.

PEG may be conjugated to the C1-inhibitor described herein directly or via a linker moiety. The polymers for conjugation to the C1-inhibitor of Formula (I) are converted into a suitably activated polymer, using the activation techniques described in U.S. Pat. Nos. 5,122,614 and 5,808,096 and other techniques known in the art without undue experimentation.

Examples of activating groups for substantially non-antigenic polymers useful for the preparation of a conjugate including polymer conjugate of Formula (I) include a list, but not limited to, aldehyde, carbonyl imidazole, chloroformate, isocyanate, PNP, tosylate, N-HOBT, and N-hydroxysuccinimidyl.

In one aspect, the activated PEG can include, but not limited to, methoxypolyethylene glycol-succinate, methoxypolyethylene glycol-succinimidyl succinate (MPEG-NHS), methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be employed in the conjugates described herein. Methods of preparing polymers having terminal carboxylic acids in high purity are described in U.S. Pat. No. 7,989,554, the content of which is incorporated herein by reference.

In alternative aspects, polymers having terminal amine groups can be employed to make the conjugates described herein. The methods of preparing polymers containing terminal amines in high purity are described in U.S. Pat. Nos. 7,868,131 and 7,569,657, the contents of each of which are incorporated by reference.

In yet a further aspect of the invention, the polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In yet a further aspect and as an alternative to PAO-based polymers such as PEG, one or more effectively non-antigenic materials such as dextran, polyvinyl alcohols, carbohydrate-based polymers, hydroxypropyl ethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will appreciate realize that the foregoing list is merely illustrative and that all polymeric materials having the qualities described herein, are contemplated. For purposes of the present invention, “substantially or effectively non-antigenic” means polymeric materials understood in the art as being nontoxic and not eliciting an appreciable immunogenic response in mammals.

### Linkers

In one aspect, the substantially non-antigenic polymer of the present invention is conjugated to C1-inhibitor via amine, amide bond or carbamate bond.

In one aspect, the substantially non-antigenic polymer of the present invention is conjugated to C1-inhibitor via linking moieties.

According to the present invention provides a polymer conjugate of Formula (1),

[PEG—(L)ₘ—(CH₂)ₙ]ₚ—(X)ₚ—C1-inhibitor (I)
wherein the bifunctional linker, L, as included in the conjugates described herein is selected from among:

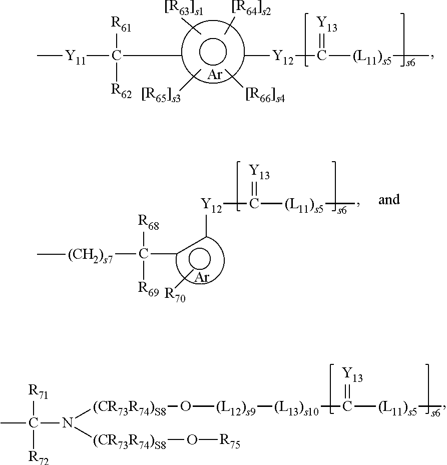

wherein
Y₁₁ is O, or S;
Y₁₂ is O, S, or NH, provided that L₁₁ is Gly-Phe-Leu-Gly, Ala-Leu-Ala-Leu, Phe-Lys, Val-Cit, when Y₁₂ is NH and (s6) is a positive integer;
Y₁₃ is O, S, or NR₆₇;
L₁₁₋₁₃ are independently bifunctional linking moiety selected from the group consisting of

—[C(═O)]ₛ₁₁CR₇₆R₇₇OCR₇₆R₇₇[C(═O)]ₛ₁₂—[Y₁₅]ₛ₁₃—;

—[C(═O)]ₛ₁₁CR₇₆R₇₇NR₇₈CR₇₆R₇₇[C(═O)]ₛ₁₂—[Y₁₅]ₛ₁₃—;

—[C(═O)]ₛ₁₁CR₇₆R₇₇SCR₇₆R₇₇[C(═O)]ₛ₁₂—[Y₁₅]ₛ₁₃—; and

—[C(═O)]ₛ₁₁(CR₇₆R₇₇)ₛ₁₁[C(═O)]ₛ₁₂—[Y₁₅]ₛ₁₃—; or

C(═Y₁₃)—L11— together form an amino acid;
Y₁₅ is O, S or NRₓ,
(s13) is 0 or a positive integer;
R₆₁, R₆₂, R₆₇, R₇₁, R₇₂, R₇₃, R₇₄ and Rₓ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, heteroalkyls, substituted C₁₋₆ heteroalkyls;
R₆₃, R₆₄, R₆₅ and R₆₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, carboxyalkyls and C₁₋₆ alkyl carbonyls;
R₆₈, R₆₉ and R₇₀ are independently selected from the group consisting of C₁₋₆ alkyls, C₁₋₁₂ branched alkyls, C₁₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
R₇₅ is H, —C(═O)—R₇₉, wherein R₇₉, in each occurrence, is the same or different alkyl,

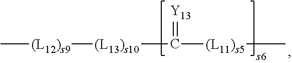

or a targeting group;

R₇₆, R₇₇ and R₇₅ are independently selected from the group consisting of from H, C₁₋₆ alkyl, alkenyl, C₂₋₆ alkynyl, C₁₋₆ heteroalkyl and aryl;
Ar is a moiety which when included in the formula forms an aromatic or heteroaromatic hydrocarbon;
(s1), (s2), (s3), and (s4) are independently zero or one;
(s5) is a positive integer of from about 1 to about 6;
(s6) is zero or a positive integer;
(s7) is zero, one or two;
(s8) is 1, 2 or 3;
(s9) is zero or one;
(s10) is zero or a positive integer of from about 1 to about 6; and
(s11), (s12), and (s13) are independently zero or one.

In a further and/or alternative embodiment, bifunctional linkers include an amino acid. The amino acid which can be selected from any of the known naturally-occurring, L-amino acids is, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof to name a few. In alternative aspects, L can be a peptide residue. The peptide can range in size, for instance, from about 2 to about 10 amino acid residues (e.g., 2, 3, 4, 5, or 6).

Derivatives and analogs of the naturally occurring amino acids, as well as various art-known non-naturally occurring amino acids (D or L form), hydrophobic or non-hydrophobic, are also contemplated to be within the scope of the invention. Simply by way of example, amino acid analogs and derivatives include:

2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyic acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine or sarcosine, N-methylisoleucine, 6-N-methyllysine, N-methylvaline, norvaline, norleucine, ornithine, and others too numerous to mention, that listed in 63 Fed. Reg., 29620, 29622 are incorporated herein by reference.

One embodiment of the L groups includes glycine, alanine, methionine or sarcosine.

Additional linkers are found in Table 1 of Greenwald et al. (Bioorganic & Medicinal Chemistry, 1998, 6:551-564 and in U.S. Pat. Nos. 6,180,095, 6,720,306, 5,965,119, 6,303,569, 6,624,142, 7,122,189, 7,897,647, 7,087,229, and 7,413,738, the contents of each of which are incorporated by reference herein.

### Synthesis of Conjugates of Formula (I)

Examples of synthesis of the polymeric conjugates of C1-inhibitor using polyethylene glycols (PEG) are provided in the following schemes.

-C1-INH Conjugation with PEG-aldehyde-

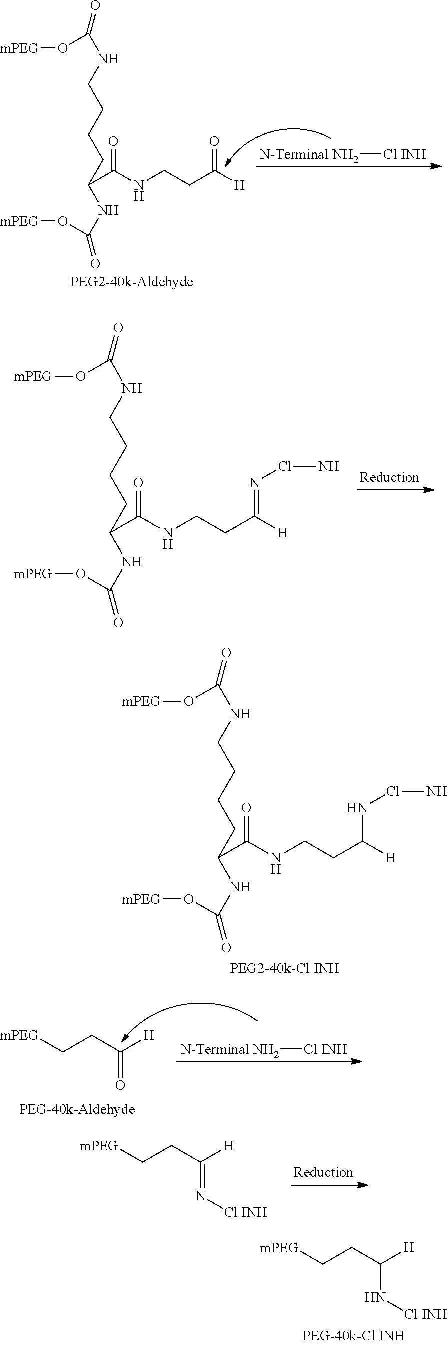

-C1 INH PEGylation with Releasable PEG-BCN3-NHS and Releasable PEG-RNL8a-NHS-

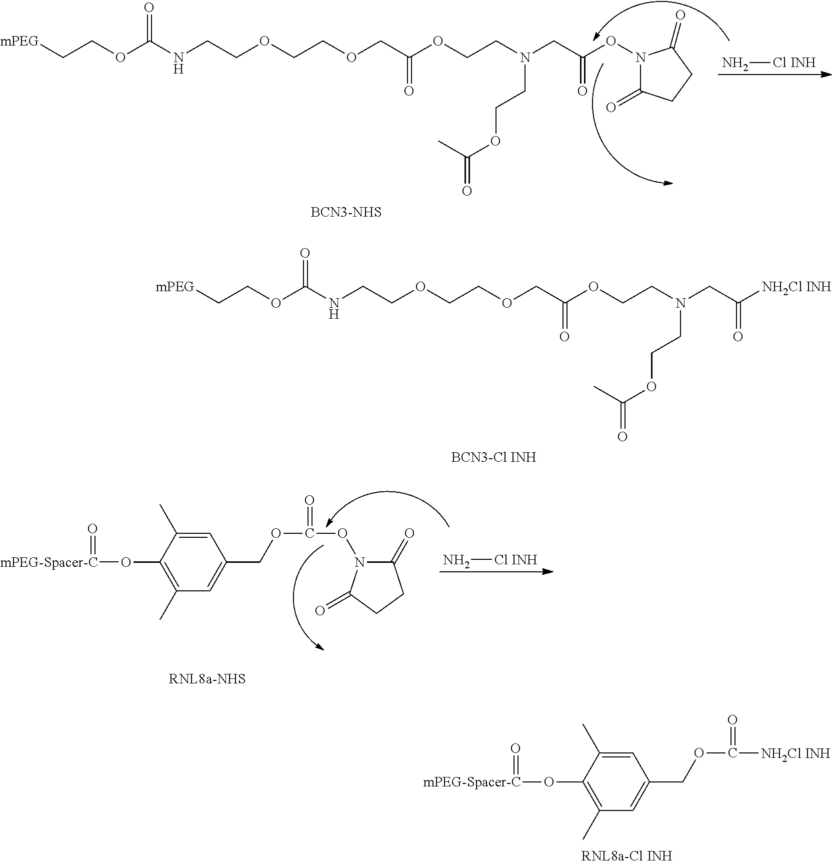

Generally, the conjugates described herein are prepared by reacting C1-inhibitor with polyalkylene oxide having an activating group, under conditions sufficient to form a covalent bond between the polyalkylene oxide and amine group of an amino acid of the C1-esterase inhibitor and purifying the resulting conjugate.

In one embodiment, the activating group is an aldehyde and the reaction is carried out in the presence of a reducing agent.

Suitable reducing agents include, for example, sodium cyanoborohydride (NaBH₃CN), sodium triacetoxyborohydride (NaBH(OC(═O)OCH₃)₃), sodium hydride, decaborane (B₁₀H₁₄), InCl₃—Et₃SiH complex, Nickel nanoparticles, Et₃SiH-iridium complex, and Ti(iOPr)₄. One preferable reducing agent is sodium cyanoborohydride,

As will be appreciated by those of ordinary skill, the aldehyde derivatives are used for N-terminal attachment of the polymer to the C1-inhibitor. For example, polyalkylene oxide (PAO) aldehydes react preferably with amines and undergo reductive amination in the presence of sodium cyanoborohydride to form a secondary or tertiary amine. Suitable polyethylene glycol (PEG) aldehydes are available from NOF and other commercial sources. Alternatively, the aldehyde can react with epsilon amine of lysine in C1-inhibitor or the secondary amine of histidine to form a tertiary amine.

In other aspects of the invention, the other activated linkers shown above will allow for non-specific linkage of the polymer to Lys amino groups-forming carbamate (urethane) or amide linkages.

In another embodiment, the activating group is selected from the group consisting of carbonyl imidazole, chloroformate, isocyanate, PNP, tosylate, N-HOST, and N-hydroxysuccinimidyl.

In some aspects of the invention, the activating group for the polymer is an oxycarbonyl-oxy-N-dicarboximide group such as a succinimidyl carbonate group. Alternative activating groups include N-succinimide, N-phthalimide, N-glutarimide, N tetrahydrophthalimide and N-norborene-2,3-dicarboxide. These urethane-forming groups are described in commonly owned U.S. Pat. No. 5,122,614, the disclosure of which is hereby incorporated by reference. Other urethane-forming activated polymers such as benzotriazole carbonate activated (BTG-activated PEG—available from Nektar) can also be used. See also commonly-assigned U.S. Pat. No. 5,349,001 with regard to the above-mentioned T-PEG.

For purposes of illustration, suitable conjugation reactions include reacting C1-inhibitor with a suitably activated polymer system described herein. The reaction is preferably carried out using conditions well known to those of ordinary skill for protein modification, including the use of a PBS buffered system, etc. with the pH in the range of about 5.0-5.5. It is contemplated that in most instances, an excess of the activated polymer will be reacted with the C1-inhibitor.

Reactions of this sort will often result in the formation of conjugates containing one or more polymers attached to the C1-inhibitor. As will be appreciated, it will often be desirable to isolate the various fractions and to provide a more homogenous product. In most aspects of the invention, the reaction mixture is collected, loaded onto a suitable column resin and the desired fractions are sequentially eluted off with increasing levels of buffer. Fractions are analyzed by suitable analytical tools to determine the purity of the conjugated protein before being processed further.

It will also be appreciated that heterobifunctional polyalkylene oxides are also contemplated for purposes of cross-linking C1-inhibitor, or providing a means for attaching other moieties such as targeting agents for conveniently detecting or localizing the polymer-C1-inhibitor conjugate in a particular areas for assays, research or diagnostic purposes.

### Formulations

Polymer conjugates of the present invention may be manufactured and formulated by processes well known in the art, e.g., using a variety of well-known mixing, dissolving, granulating, levigating, emulsifying, encapsulating, entrapping or lyophilizing processes. Compositions may be formulated in conjunction with one or more physiologically acceptable carriers comprising excipients and auxiliaries which facilitate processing of the active conjugates into preparations which can be used pharmaceutically. Proper formulation is dependent upon the route of administration chosen. Parenteral routes are preferred in many aspects of the invention, but not limited to.

In another aspect, the conjugates may also be formulated for parenteral administration or injection, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. For injection, including, without limitation, intravenous, intramuscular and subcutaneous injection, the polymer conjugates of the invention may be formulated in aqueous solutions, preferably in physiologically compatible buffers such as physiological saline buffer or polar solvents including, without limitation, a pyrrolidone or dimethylsulfoxide. Aqueous injection suspensions may contain substances that increase the viscosity of the suspension, such as sodium carboxymethyl cellulose, sorbitol, or dextran. Additionally, suspensions of the active conjugates may be prepared in a lipophilic vehicle. Suitable lipophilic vehicles include fatty oils such as sesame oil, synthetic fatty acid esters such as ethyl oleate and triglycerides, or materials such as liposomes. Optionally, the suspension may also contain suitable stabilizers and/or agents that increase the solubility of the conjugates to allow for the preparation of highly concentrated solutions. Alternatively, the active ingredient may be in powder form, such as lyophilized product, for constitution with a suitable vehicle, e.g., sterile, pyrogen-free water, before use.

### Methods of Administration and Dosage

The C1-inhibitor polymer conjugate described herein is useful for all of the methods and indications already art-known for Cinryze® (Viro Pharma Biologics, Inc.) and Berinert® (CSL Behring LLC). Thus, the inventive C1-inhibitor conjugate is administered to a patient in need thereof in an amount that is effective to treat a disease or disorder or other condition that is responsive to such treatment. The artisan will appreciate suitable amounts, routes of administration and dosing schedules extrapolated from the known properties of Cinryze® and Berinert®.

Another aspect of the present invention provides methods of treatment for various medical conditions in mammals, preferably humans. The methods include administering an effective amount of a pharmaceutical composition that includes a C1-inhibitor polymer conjugate prepared as described herein, to a mammal in need of such treatment. The conjugates are useful for, among other things, treating C1-inhibitor-susceptible conditions or conditions which would respond positively or favorably as these terms are known in the medical arts to C1-inhibitor-based therapy.

Conditions that can be treated in accordance with the present invention are generally those that are susceptible to treatment with C1-inhibitor. Exemplary conditions which can be treated with C1-inhibitor include, but are not limited to, ongoing, acute attacks of hereditary angioedema (HAE) affecting the abdomen, face or throat in adults and adolescents and all other medical conditions know to those of ordinary skill to benefit from C1-inhibitor therapy. In a preferred aspect of the invention, the polymer conjugated C1-inhibitor is administered to patients in amounts effective to treat hereditary angioedema or prevent swelling and/or painful attacks in teenagers and adults with Hereditary Angioedema.

Administration of the described dosages may be every other day, but is preferably once or twice a week. Doses are usually administered over at least a 24 week period by injection or infusion. Administration of the dose can be intravenous, subcutaneous, intramuscular, or any other acceptable systemic method, including subdermal or transdermal injection via conventional medical syringe and/or via a pressure system. Based on the judgment of the attending clinician, the amount of drug administered and the treatment regimen used will, of course, be dependent on the age, sex and medical history of the patient being treated, the stage or severity of the specific disease condition and the tolerance of the patient to the treatment as evidenced by local toxicity and by systemic side-effects. Dosage amount and frequency may be determined during initial screenings of neutrophil count.

The amount of the C1-inhibitor polymer conjugate composition administered to treat the conditions described above is based on the C1-inhibitor activity of the polymeric conjugate. It is an amount that is sufficient to significantly affect a positive clinical response. Although the clinical dose will cause some level of side effects in some patients, the maximal dose for mammals including humans is the highest dose that does not cause unmanageable clinically-important side effects. For purposes of the present invention, such clinically important side effects are those which would require cessation of therapy due to severe flu-like symptoms, central nervous system depression, severe gastrointestinal disorders, alopecia, severe pruritus or rash. Substantial white and/or red blood cell and/or liver enzyme abnormalities or anemia-like conditions are also dose limiting.

A therapeutically effective amount refers to an amount of conjugate effective to prevent, alleviate or ameliorate the C1-inhibitor-susceptible condition. Determination of a therapeutically effective amount is well within the capability of those skilled in the art, especially in light of the disclosure herein.

The dosage, of course, can vary depending upon the dosage form and route of administration. The exact formulation, route of administration and dosage can be selected by the individual physician in view of the patient's condition.

For any conjugate used in the methods of the invention, the therapeutically effective amount may be estimated initially from in vitro assays. Then, the dosage can be formulated for use in animal models so as to achieve a circulating concentration range that includes the effective dosage. Such information can then be used to more accurately determine dosages useful in patients.

Toxicity and therapeutic efficacy of the conjugates described herein can be determined by standard pharmaceutical procedures in cell cultures or experimental animals using methods well-known in the art.

As explained above, the dosages of the polymer C1-inhibitor conjugate compositions of the present invention will vary somewhat depending upon the C1-inhibitor moiety and polymer selected. In general, however, the conjugate is administered in amounts ranging from about 100 to about 5,000 u/kg/week, from about 500 to about 4,000 u/kg/week or from about 1,000 to 3,000 u/kg/week of C1-inhibitor equivalent in the polymer conjugate, based on the condition of the treated mammal or human patient. The range set forth above is illustrative and those skilled in the art will determine the dosing of the conjugate selected based on clinical experience and the treatment indication.

The conjugates may be administered once daily or divided into multiple doses which can be given as part of a multi-week treatment protocol. The precise dose will depend on the stage and severity of the condition, the susceptibility of the condition to the C1-inhibitor polymer conjugate, and the individual characteristics of the patient being treated, as will be appreciated by one of ordinary skill in the art.

Practice of the invention would allow treatment of this condition, and others, at higher doses and in combination with other art-known therapeutic agents.

### EXAMPLES

The following examples serve to provide further appreciation of the invention but are not meant in any way to restrict the effective scope of the invention.

### Materials

Reagents: C1 Esterase inhibitor was obtained from Athens Research & Technology and has MW: 73000 Da as determined by MALDI. ALD-PEG-40k was obtained from NOF;
Buffers (1) 100 mM Na acetate, 150 mM naCl, pH5.5; (2) PBS
Ultrafiltration: 10 k Pellicon XL 50 Ultrafiltration Cassettes
Amicon Membrane: 30K Ultrafiltration Membrane (Millipore)
IP Sterile Filter: 0.2 μm sterile polyethersulfone filter (VWR)

### Characterization of PEG-C1 INH

The concentration of PEGylated C1 INH was determined by UV at 280 nm. The Sample at 5 μg or 10 μg was loaded into the gel without sample reduction and heating for electrophoresis (Novex NuPAGE 10% Bis-Tris gel, Invitrogen). The protein bands were visualized after simple blue stain. The density of the image was obtained on Molecular Dynamics. As seen on SDS gel, all C1 was converted into PEGylated form.

### Example 1

### C1-INH PEGylation with ALD-PEG-40k and ALD-PEG2-40k

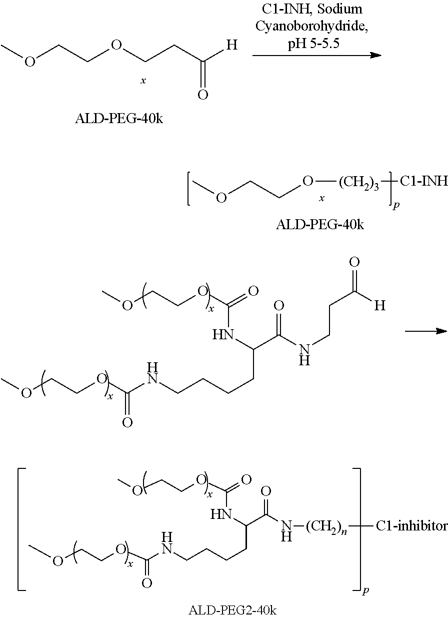

Human C1-esterase inhibitor, C1-INH, was suspended in a 100 mM sodium acetate buffer at pH 5.0-5.5 in 1.5 mg/ml concentration. To the suspension, PEG aldehyde, ALD-PEG-40k or ALD-PEG2-40k, was added at 10-15:1 reaction molar ratio of PEG to C1-INH in the presence of sodium cyanoborohydride. The concentration of sodium cyanoborohydride was kept at 15 mM and the reaction was conducted at 20° C. for 16 hours. The conjugates were purified as mentioned above using standard chromatogram purification techniques.

### Example 2

### 5k mPEG-BCN-C1 INH

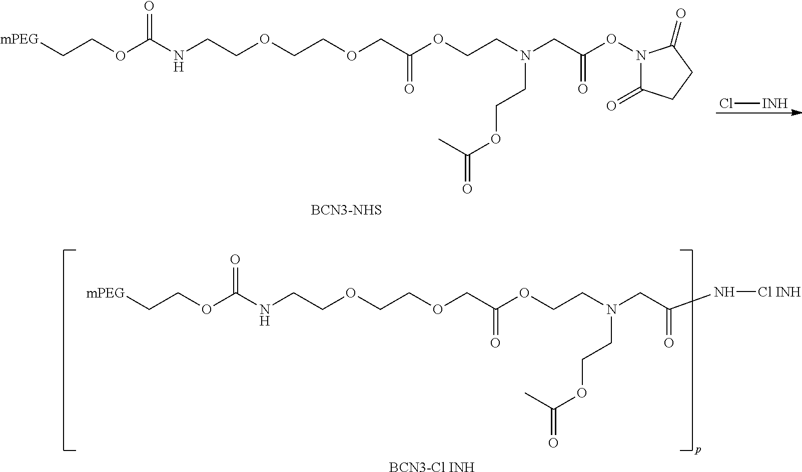

50 mg of native C1 inhibitor was diluted by 12 ml of 100 mM Na phosphate, pH7.0, resulting 15.6 ml. Dissolved 1.4 g of 5k mPEG-BCN3-NHS in 9.4 ml of 0.1 M Na phosphate, pH 7.0 (150 mg/ml). Mixed 5k mPEG-BCN-PEG and C1 inh together (mole ratio PEG:C1 inh=50:1) by a stirring bar at 500 rpm. Left the reaction solution at room temperature (23° C.) for 2 hrs with stirring. Free PEG was removed by a TFF LabScale™ (Millipore, MA) equipped by one 10 k Pellicon XL 50 Ultrafiltration Cassette (Millipore) pre-equilibrated by 100 mM Na phosphate at 6.80 in a cold room. Free PEG in permeate was monitor by RP-HPLC analysis after 20 volumes of diafiltration against the buffer of 100 mM Na phosphate at 6.80 in a cold room. At the end of diafiltration, switch the diafiltration buffer to PBS (pH7.4) and continued the diafiltration to pH7.4 monitored by the pH of the permeate. Stopped the diafiltration when the pH of the permeate reached 7.4. The sample was drained out of the system with 2 rinses (30 ml each rinse), resulting 80 ml of combined sample. The sample was further concentrated in an Amcicon® 8050 installed with one piece of 10 K Ultrafiltration Membrane (Millipore) in a cold room to ˜5 ml. Pipetted the sample out and rinsed the membrane by 5 ml of PBS. Combined the sample with the rinse in a tube, resulting 10 ml at 4.0 mg/ml. Filtered this sample by a 0.2 μm sterile polyethersulfone filter (VWR). The conjugates were purified as mentioned above using standard chromatogram purification techniques. Protein Cone by A280 was 4.0 mg/ml and C1 in activity was 3.7 U/mg. Free PEG was not detected by RP-HPLC or Native C1 inh was not observed by SDS-PAGE.

### Example 3

### 5k mPEG-RNL8a C1 INH

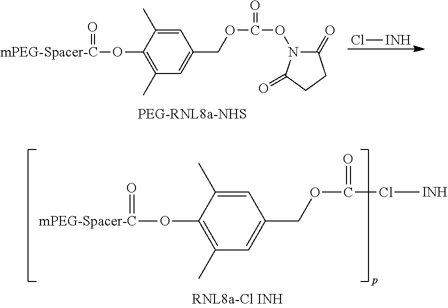

40 mg of native C1 inhibitor was diluted by 12 ml of 100 mM Na phosphate, pH7.0, resulting 12.84 ml. Dissolved 1.4 g of 5k mPEG-RNL8a in 9.4 ml of 0.1 M Na phosphate, pH 7.0 (150 mg/m1) by a stirring bar at 500 rpm to completely dissolve. Mixed PEG and C1 inh together (mole ratio PEG:C1 inh=50:1) by a stirring bar at 500 rpm. Left the reaction solution at room temperature (23° C.) for 2 hrs with stirring. Free PEG was removed by a TFF LabScale™ (Millipore, MA) equipped by one 10k Pellicon XL 50 Ultrafiltration Cassette (Millipore) pre-equilibrated by 100 mM Na phosphate at pH6.80 in a cold room. Free PEG in the permeate was monitor by RP-HPLC after 20 volumes of diafiltration against the buffer of 100 mM Na phosphate at pH6.80 in a cold. room. At the end of diafiltration, switch the buffer to PBS (pU7.4) and continued the diafiltration to pH7.4 monitored by the pH of permeate. Stopped the diafiltration when the pH of permeate reached 7.4, The sample was drained out of the system with 2 rinses (30 ml each rinse), resulting 80 ml. The sample was further concentrated in an Amcicone® 8050 installed with one piece of 10 K Ultrafiltration Membrane (Millipore) in a cold room to 5 ml. Pipetted the sample out and rinsed the membrane by 5 ml of PBS (pH7.4). Combined the sample with the rinse in a tube, resulting 9 ml. Filtered this sample by a 0.2 μm sterile polyethersulfone filter (VWR). The conjugates were purified as mentioned above using standard chromatogram purification techniques. Protein Conc by A280 was 5.50 mg/ml and C1 in activity was 3.8 U/mg. Free PEG was not detected by RP-HPL and Native C1 inh was not observed by SDS-PAGE.

### Example 4

### Purification of Mono and Di PEGylated C1 INH Conjugates

Mono or Di PEGylated C1-INH (both PEG linear and branched) was purified by weak anion exchange column (HiTrap DEAE FF, 1 ml. GE Healthcare) or by hydrophobic interaction column (HIC phenyl FF, 1 ml, GE Healthcare).

In DEAE column purification, Buffer A contained 10 mM Tris, pH 8.5 and buffer B had 0.5 M NaCl in buffer A. Elution was conducted at 1 ml/min over 30 min. Based on SDS-PAGE, the majority components in flow through was di PEG-C1 INH. Mono PEG-C1 INH and native C1 INH were both bound to the column and started to elute out at ˜0.12 M NaCl. The fractions containing mono PEG-C1 INH identified by SDS-PAGE was concentrated using Centricon YM30 (Millipore) and the buffer was exchanged to PBS by NAP-5 column (GE Healthcare).

In HIC phenyl purification, Buffer A contained 0.75 M ammonium sulfite in PBS buffer and buffer was PBS. Elution was conducted at 1 ml/min over 30 min. The first elution peak. identified on SDS-PAGE was mono PEG-C1 INH and second peak was di PEG-C1 INH. Mono and di PEG-C1 INH were concentrated using Centricon YM30 and buffer-exchanged to PBS by NAP-5 column. The conjugates were purified as mentioned above using standard chromatogram purification techniques.

### Example 5

### Composition of PEGylated C1 INH by SDS-PAGE

The concentrations of mono or di PEGylated C1 INH were determined by UV at 280 nm. 1.5-μg protein was loaded into the gel without sample reduction and heating (Novex NuPAGE 4-12% Bis-Tris gel, Invitrogen). The electrophoresis was conducted at 200 Voltage for 30 mM and the protein bands were visualized after simple blue stain. The density of the image was obtained on Molecular Dynamics.

### Example 6

### C1-INH Activity Assay

C1 INH activity was measured by the inhibition of C1 esterase activity. Samples, standards, and controls were added to 96-well plate, and then C1-esterase was added. After 10 min incubation at 37° C., substrate was added. C1-esterase activity for cleavage of the substrate was monitored at 37° C. for 4 minutes kinetically. The higher C1 INH activity results in the lower C1 esterase activity or the lower kinetics of substrate cleavage.

| | PEG#/ | Specific Activity | % C1 |
|---|---|---|---|
| Sample | C1 INH | (U/mg) | INH Activity |
| ALD-PEG(40 k)-C1 INH | 1 | 6.1 | 67 |
| ALD-PEG(40 k)-C1 INH | 1 | 6.9 | 76 |
| ALD-PEG(40 k)-C1 INH | 2 | 6.8 | 75 |
| ALD-PEG2(40 k)-C1 INH | 1 | 7.4 | 81 |
| ALD-PEG2(40 k)-C1 INH | 2 | 3.9 | 43 |
| 5 kmPEG-BCN-C1 INH | 8-9 | 7.3 | 100% |
| 5 kmPEG-RNL8a C1 INH | 15 | 3.8 | 53% |

The C1 esterase inhibitor protein has to bind to another enzyme to have activity. Thus, indiscriminate chemical modification could result in complete loss or significant reduction of biological activity.

The polymer conjugate of the present invention, as measured above, retained significant amount of C1-esterase inhibitor activity. The first mono PEGylation on the N-terminal retained 67-81% of the C1-esterase inhibitor activity. Even PEGylation of the less selective Lysine, which could be near the C-terminal, also allowed the polymer conjugate to retain 43 or 75% of the C1-esterase inhibitor activity. It was a surprising result because it was speculated that modification of the active domain, C-terminal, can reduce the activity dramatically. Without being bound to any theory, it is possible that the present PEG attached to the Lysine was still flexible enough to provide freedom for C-terminal for the high inhibitory activity. The above results provide that PEGylation of the present invention did not alter the C1-esterase activity even after the second PEGylation.

### Example 7

### In vivo Pharmacokinetics

The polymeric conjugates of C1 inhibitor prepared was administered (i.v.) to groups of rat for in vivo plasma pharmacokinetic (PK) study at dose of 70 U/kg. The polymer conjugates of the invention such as ALD-PEG-C1 INH demonstrated improved half-lives compared to the native C1-esterase inhibitor. Some polymer conjugate extended half-life to about 77 hours, with more than 10 folds improvement than the native C1 inhibitor. This profile can provide a long lasting treatment regime such as once a week.

1. A polymer conjugate, comprising:
a C1-inhibitor having at least one substantially non-antigenic polymer covalently attached thereto via amino group of the C1-inhibitor.

2. The polymer conjugate of claim 1, wherein the substantially non-antigenic polymer is a polyalkylene oxide.

3. The polymer conjugate of claim 2, wherein the polyalkylene oxide is PEG

4. The polymer conjugate of claim 1, wherein the C1-inhibitor is a human C1 esterase inhibitor (C1-INH).

5. The polymer conjugate of claim 1, wherein the C1-inhibitor is a polypeptide represented by SEQ ID NO:1 or SEQ ID NO: 2.

6. The polymer conjugate of claim 1, wherein one of the substantially non-antigenic polymer is attached to the N-terminal, an epsilon amino group of a lysine, or histidine of the C1 inhibitor.

7-9. (canceled)

10. The polymer conjugate of claim 1, wherein the polymer conjugate retains about 60-80% of the biological activity of the C1-inhibitor in its native form.

11. (canceled)

12. The polymer conjugate of claim 1, wherein the molecular weight of the substantially non-antigenic polymer ranges from about 2,000 to about 100,000 daltons.

13. The polymer conjugate of claim 1, wherein the substantially non-antigenic polymer is conjugated via amine, amide bond or carbamate bond.

14. The polymer conjugate of claim 3, wherein the conjugate comprises Formula (I):
[PEG—(L)ₘ—(CH₂)ₙ]ₚ—(X)_{p′}—C1-inhibitor (I)
wherein
PEG is linear, branched or multi-arm PEG having terminal group —(CH₂CH₂O)—;
L is a linker;
(m) is 0 or 1;
(n) is zero or a positive integer;
(p) is a positive integer; and
X is an amine group of an amino acid found on C1 inhibitor attached to the polymer; (p′) is a positive integer same as (p), provided that (m) and (n) are not zero simultaneously.

15. (canceled)

16. The polymer conjugate of claim 14, wherein L is selected from the group consisting of: 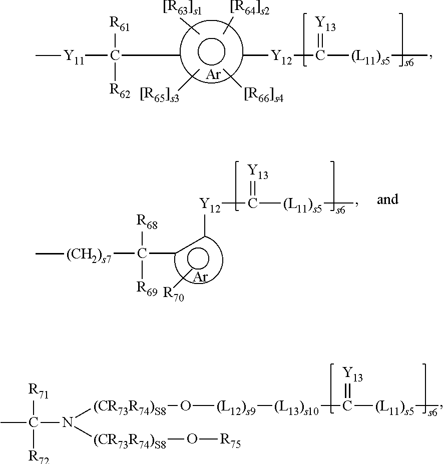
wherein
Y₁₁ is O, or S;
Y₁₂ is O, S, or NH, provided that L₁₁ is Gly-Phe-Leu-Gly, Ala-Leu-Ala-Leu, Phe-Lys, or Val-Cit, when Y₁₂ is NH and (s6) is a positive integer;
Y₁₃ is O, S, or NR₆₇;
L₁₁₋₁₃ are independently bifunctional linking moiety selected from the group consisting of
—[C(═O)]ₛ₁₁CR₇₆R₇₇OCR₇₆R₇₇[C(═O)]ₛ₁₂—[Y₁₅]ₛ₁₃—;
—[C(═O)]ₛ₁₁CR₇₆R₇₇NR₇₈CR₇₆R₇₇[C(═O)]ₛ₁₂—[Y₁₅]ₛ₁₃—;
—[C(═O)]ₛ₁₁CR₇₆R₇₇SCR₇₆R₇₇[C(═O)]ₛ₁₂—[Y₁₅]ₛ₁₃—; and
—[C(═O)]ₛ₁₁(CR₇₆R₇₇)ₛ₁₁[C(═O)]ₛ₁₂—[Y₁₅]ₛ₁₃—; or
C(═Y₁₃)—L11— together form an amino acid;
Y₁₅ O, S or NRₓ;
(s13) is 0 or a positive integer;
R₆₁, R₆₂, R₆₇, R₇₁, R₇₂, R₇₃, R₇₄ and Rₓ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls;
R₆₃, R₆₄, R₆₅ and R₆₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;
R₆₈, R₆₉ and R₇₀ are independently selected from the group consisting of C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
R₇₅ is H, —C(═O)—R₇₉, wherein R₇₉, in each occurrence, is the same or different alkyl, 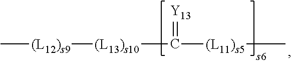
or a targeting group;
R₇₆, R₇₇ and R₇₈ are independently selected from the group consisting of from H, C₁₋₆ alkyl, C₂₋₆ alkenyl, C₂₋₆ alkynyl, C₁₋₆ heteroalkyl and aryl;
Ar is a moiety which when included in the formula forms an aromatic or heteroaromatic hydrocarbon;
(s1), (s2), (s3), and (s4) are independently zero or one;
(s5) is a positive integer of from about 1 to about 6;
(s6) is zero or a positive integer;
(s7) is zero, one or two;
(s8) is 1, 2 or 3;
(s9) is zero or one;
(s10) is zero or a positive integer; and
(s11), (s12), and (s13) are independently zero or one.

17. The polymer conjugate of claim 3 selected from the group consisting of: 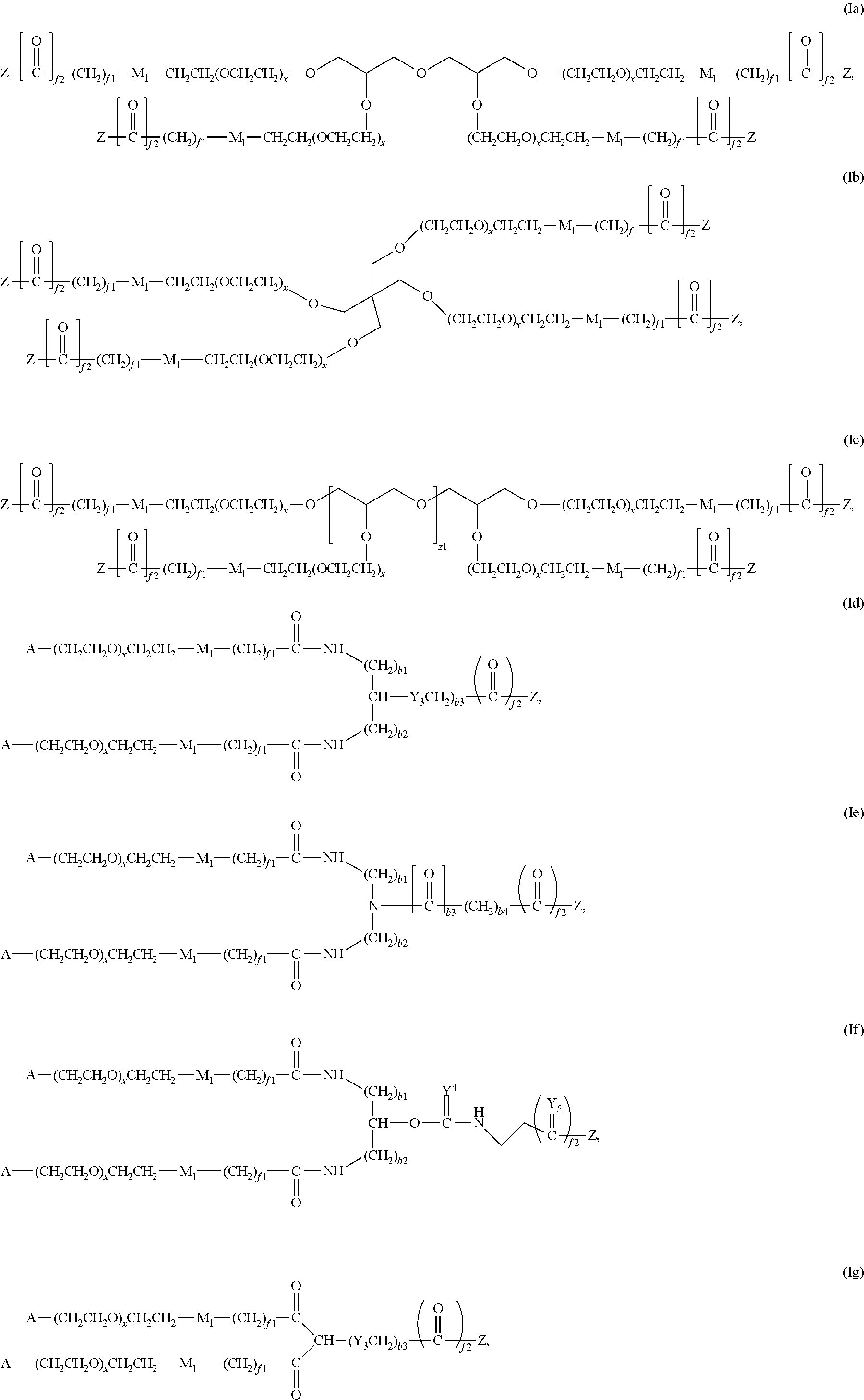 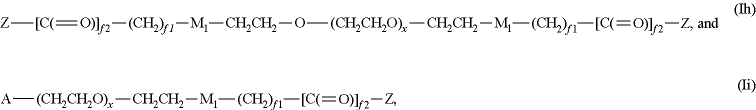
wherein
A is hydroxyl, NH₂, CO₂H, or C₁₋₆ alkoxy;
M₁ is O, S, or NH;
Y₃ is O, NR₅₁, S, SO or SO₂;
Y₄ and Y₅ are independently O, S or NR₅₁;
R₅₁, in each occurrence, is independently hydrogen, C₁₋₈ alkyl, C₁₋₈ branched alkyl, C₁₋₈ substituted alkyl, aryl, or aralkyl;
Z, in each occurrence, is independently OH, a leaving group, a targeting group, C₁₋₈ alkyl, C₁₋₈ alkoxy, C1 inhibitor or C1 inhibitor containing moiety;
(b1) and (b2) are independently zero or positive integers;
(b3) is zero or 1;
(b4) is a positive integer;
(f1) is zero or a positive integer of from about 1 to about 10;
(f2) is zero or 1;
(z1) is zero or a positive integer of from 1 to about 27;
(x) is a degree of polymerization positive integer of from about 10 to about 2,300 so that the polymeric portion of the compound has the total number average molecular weight of from about 2,000 to about 100,000 daltons, provided that one or more Z are C1 inhibitors or C1 inhibitor containing moiety.

18. The polymer conjugate of claim 3 selected from the group consisting of: 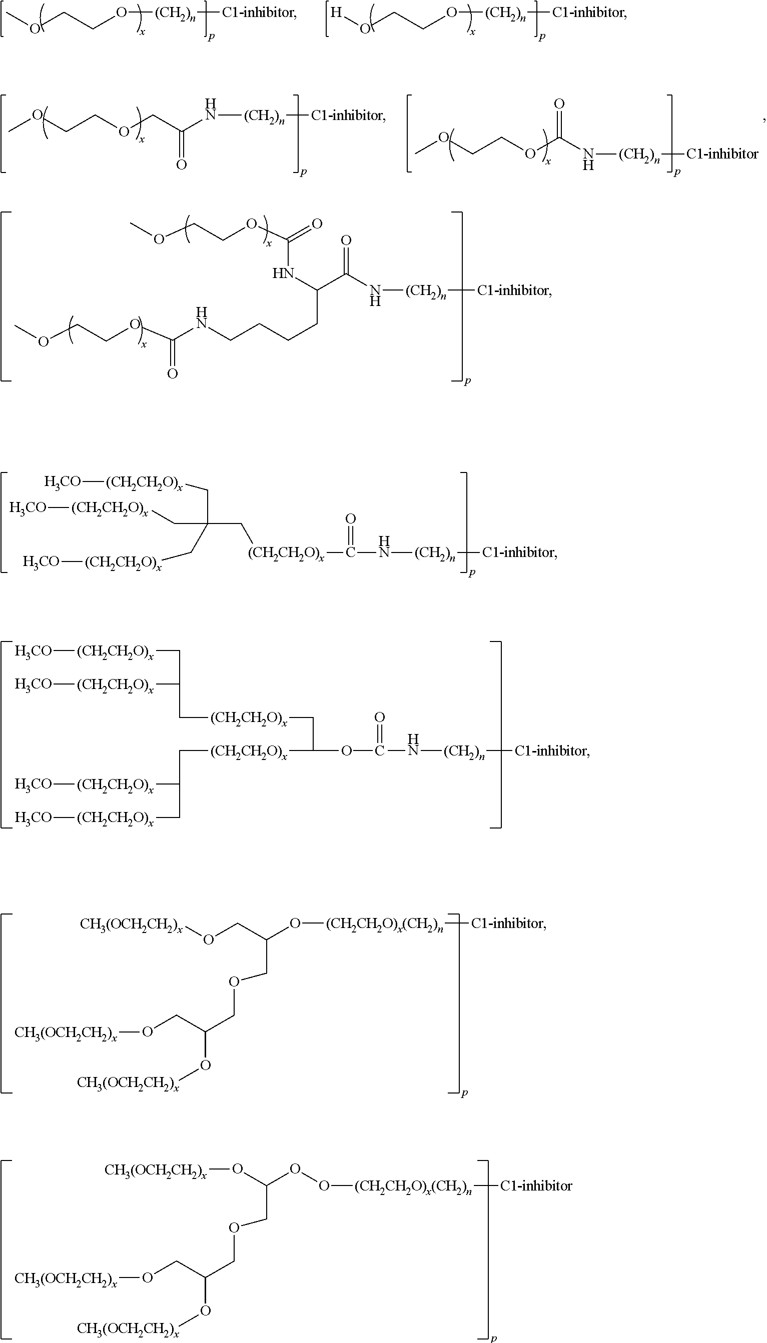 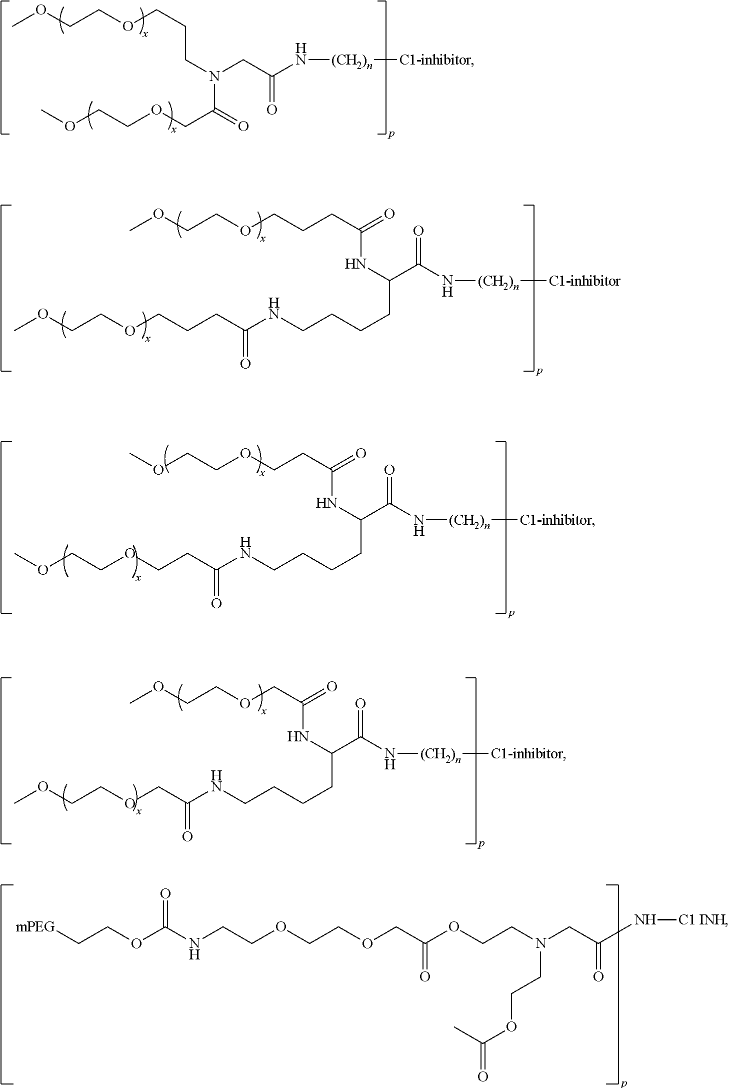 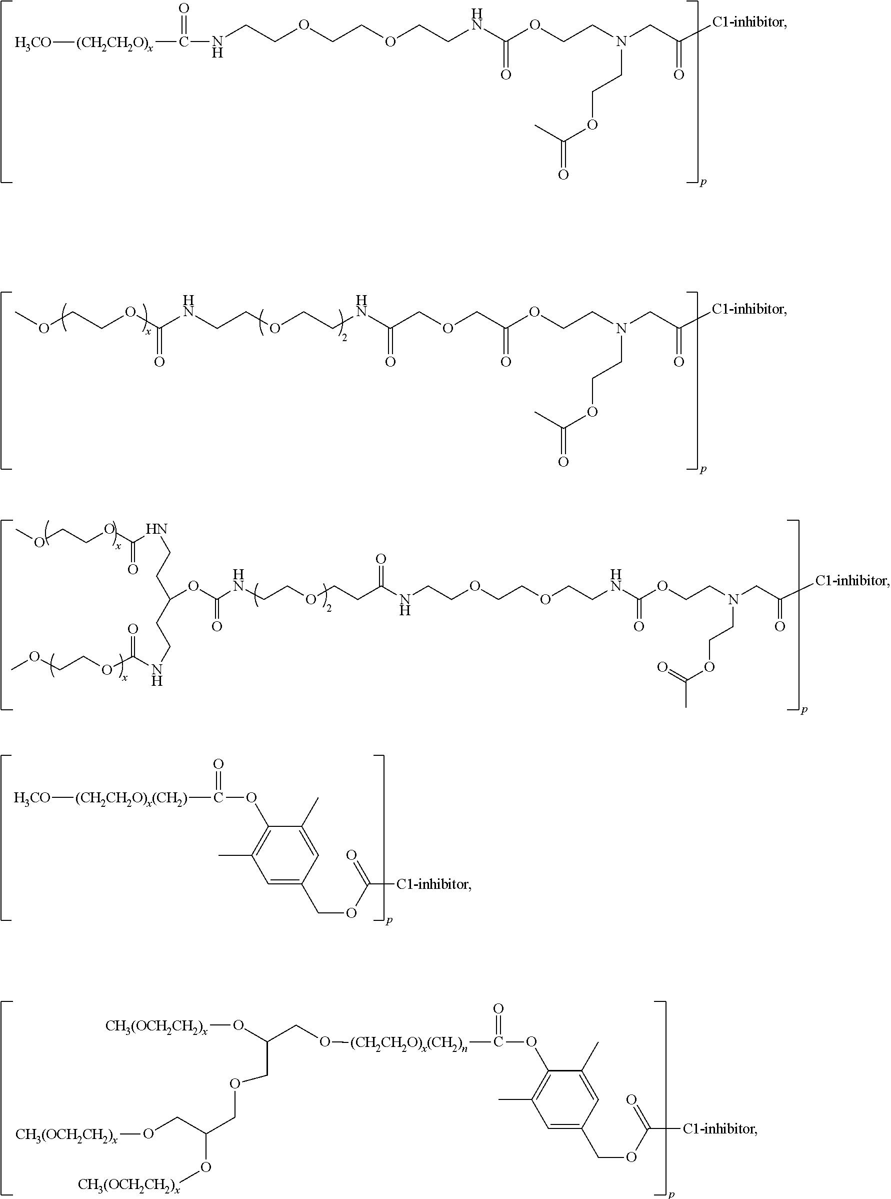 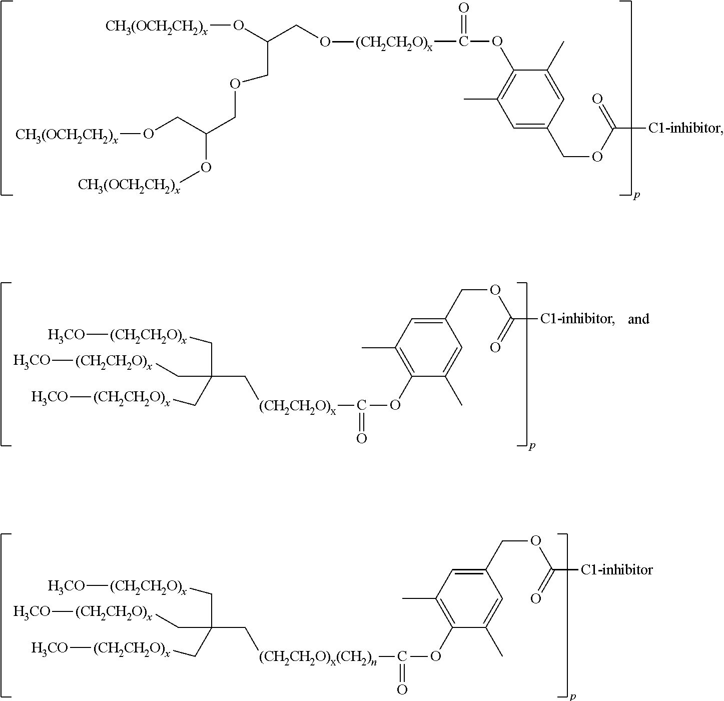
wherein,
C1-inhibitor is bonded via nitrogen of amino acids;
(x) is a degree of polymerization positive integer of from about 10 to about 2,300 so that the polymeric portion of the compound has the total number average molecular weight of from about 2,000 to about 100,000 daltons;
(n) is zero or a positive integer; and
(p) is a positive integer.

19. A method of preparing a the polymer conjugate comprising a C1-esterase inhibitor having at least one polyalkylene oxide attached thereto via an amino group of the C1-esterase inhibitor, the method comprising:
reacting C1-esterase inhibitor with a polyalkylene oxide having an activating group, under conditions sufficient to form a covalent bond between the polyalkylene oxide and amine group of an amino acid of the C1-esterase inhibitor; and
purifying the resulting conjugate.

20. The method of claim 19, wherein the activating group is an aldehyde and the reaction is carried out in the presence of a reducing agent.

21. The method of claim 19, wherein the activating group is selected from the group consisting of carbonyl imidazole, chloroformate, isocyanate, PNP, tosylate, N-HOBT, and N-hydroxysuccinimidyl.

22. A method of treating a mammal comprising administering an effective amount of a polymer conjugate of claim 1 to patient in need thereof.

23. The method of claim 21, wherein the polymer conjugate is administered in amounts from about 100 u/kg/week to about 5,000 u/kg/week of C1-inhibitor equivalent in the polymer conjugate.

24. The method of claim 21, wherein the polymer conjugate is administered in amounts from about 500 u/kg/week to about 4000 u/kg/week of C1-inhibitor equivalent in the polymer conjugate.

25. The polymer conjugate of claim 4, wherein the conjugate is represented by the formula: 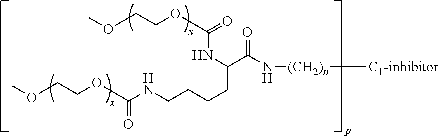
wherein,
the C1 esterase inhibitor is a polypeptide represented by SEQ ID NO:1 or SEQ ID NO:2 and is bonded via nitrogen of amino acids;
(x) is a degree of polymerization positive integer of from about 10 to about 2,300 so that the polymeric portion of the compound has the total number average molecular weight of from about 2,000 to about 100,000 daltons;
(n) is 3; and
(p) is 1.
